(19) United States
(12) Patent Application Publication
(10) Pub. No.: US 2023/0207125 A1
(43) Pub. Date: Jun. 29, 2023

(54) DIAGNOSIS-ADAPTIVE PATIENT ACUITY MONITORING

(71) Applicant: KONINKLIJKE PHILIPS N.V., EINDHOVEN (NL)

(72) Inventors: Kristen TGAVALEKOS, CAMBRIDGE, MA (US); Claire Yunzhu ZHAO, BOSTON, MA (US); Shreyas RAVINDRANATH, PHOENIX, AZ (US)

(21) Appl. No.: 17/917,585

(22) PCT Filed: Apr. 9, 2021
(86) PCT No.: PCT/EP2021/059255

§ 371 Date: Oct. 7, 2022

Related U.S. Application Data
(60) Provisional application No. 63/008,092, filed on Apr. 10, 2020.

(51) Int. Cl. G16H 50/20; G16H 10/60

(52) U.S. Cl. CPC G16H 50/20; G16H 10/60

(57) ABSTRACT

A non-transitory computer readable medium (26) stores instructions executable by at least one electronic processor (20) to perform a method (100) of acuity monitoring of a patient. The method includes: generating a set of diagnosis-specific acuity scores (32) for a plurality of diagnoses using diagnosis-specific acuity scoring modules (28) for the respective diagnoses of the plurality of diagnoses applied to clinical metrics of the patient; determining at least one primary diagnosis (34) of the patient using a computer-aided diagnosis (CAD) module (30) applied to the clinical metrics of the patient; selecting an acuity score for the at least one primary diagnosis from the set of diagnosis-specific acuity scores; and displaying an indication of the at least one primary diagnosis and the acuity score for the at least one primary diagnosis.

### FIELD

The following relates generally to the patient monitoring arts, vital sign monitoring arts, patient acuity or status assessment arts, patient care quality arts, patient workflow optimization arts, and related arts.

### BACKGROUND

To create a unified understanding of a patient status, physicians view and link together patient data from multiple disparate sources. For example, charted vital signs, lab results, waveforms from bedside monitoring, and imaging examination notes all contain patient information, and it is often left up to the care team to synthesize this information into a clinically actionable understanding of the medical state of the patient so they may optimize treatment plans and safe hospital transitions. Ineffective hospital transitions can lead to poor utilization of hospital resources and high readmission rates. Existing metrics of patient status such the Modified Warning Score (MEWS) (see, e.g., C. P. Subbe, “Validation of a modified Early Warning Score in medical admissions,” QJM, vol. 94, no. 10, pp. 521-526, October 2001) and Early Deterioration Indicator (EDI) (see, e.g., E. Ghosh, L. Eshelman, L. Yang, E. Carlson, and B. Lord, “Early Deterioration Indicator: Data-driven approach to detecting deterioration in general ward,” Resuscitation, vol. 122, pp. 99-105, January 2018) operate by scoring patient illness acuity according to features such as vital signs, with higher scores indicating higher risk of deterioration. These metrics provide a summary of data from different sources in order to support the care team in their clinical decisions.

However, a general-purpose patient status metric such as MEWS does not take into account the primary (much less secondary) diagnoses of patients. The subset of key physiologic measurements and their acceptable range of values can vary according to diagnosis or disease. To address this, patient status metrics have been developed specifically for acute heart failure (AHF) patients that predict inpatient mortality, such as the Acute Decompensated Heart Failure National Registry (ADHERE) model and the Get with the Guidelines Heart Failure (GWTG-HF) model (see, e.g., T. Lagu et al., “Validation and Comparison of Seven Mortality Prediction Models for Hospitalized Patients With Acute Decompensated Heart Failure,” Circ. Heart Fail., vol. 9, no. 8, August 2016). Acute kidney injury (see, e.g., T.-Y. Tsai et al., “Comparison of RIFLE, AKIN, and KDIGO classifications for assessing prognosis of patients on extracorporeal membrane oxygenation,” J. Formos. Med. Assoc., vol. 116, no. 11, pp. 844-851, November 2017) is an additional example of a diagnosis that has a specific acuity score. While acuity scores designed for a specific diagnosis may be more informative than general-purpose acuity score models based on a heterogeneous population, use of a diagnosis-specific patient status metric presupposes accurate identification of each patient’s diagnosis, which may be challenging to identify or may change over time. For example, many patients suffer from multiple conditions, e.g. a patient may have AHF and also be at high risk for acute kidney injury (AKI), and may have a further underlying disease such as type II diabetes. In this illustrative patient, it may not be apparent whether an AHF-specific acuity score or an AKI-specific acuity score is more appropriate, and furthermore the most appropriate diagnosis -specific acuity score may change over time.

The following discloses certain improvements to overcome these problems and others.

### SUMMARY

In one aspect, a non-transitory computer readable medium stores instructions executable by at least one electronic processor to perform a method of acuity monitoring of a patient. The method includes: generating a set of diagnosis-specific acuity scores for a plurality of diagnoses using diagnosis-specific acuity scoring modules for the respective diagnoses of the plurality of diagnoses applied to clinical metrics of the patient; determining at least one primary diagnosis of the patient using a computer-aided diagnosis (CAD) module applied to the clinical metrics of the patient; selecting an acuity score for the at least one primary diagnosis from the set of diagnosis-specific acuity scores; and displaying an indication of the at least one primary diagnosis and the acuity score for the at least one primary diagnosis.

In another aspect, a method of acuity monitoring of a patient includes: generating a set of diagnosis-specific acuity scores for a plurality of diagnoses using diagnosis-specific acuity scoring modules for the respective diagnoses of the plurality of diagnoses applied to clinical metrics of the patient; determining at least one primary diagnosis and at least one secondary diagnosis of the patient using a CAD module applied to the clinical metrics of the patient; selecting an acuity score for the at least one primary diagnosis and for each secondary diagnosis from the set of diagnosis-specific acuity scores; determining diagnosis probabilities for the at least one primary diagnosis and the at least one secondary diagnosis using the CAD module applied to the clinical metrics of the patient; and displaying an indication of the at least one primary diagnosis, an indication of each secondary diagnosis, the acuity score for the at least one primary diagnosis and each secondary diagnosis, and indications of the corresponding diagnosis probabilities.

In another aspect, an acuity monitoring apparatus for monitoring acuity of a patient includes: a data input module configured to acquire and/or receive clinical metrics of the patient, and a display device. An electronic processor is programmed to: implement diagnosis-specific acuity scoring modules for respective diagnoses of a plurality of diagnoses; implement a CAD module; and iteratively perform an acuity monitoring method applied to most recent clinical metrics of the patient acquired and/or received by the data input module. The acuity monitoring method includes: generating a set of diagnosis-specific acuity scores by applying the diagnosis-specific acuity scoring modules to the most recent clinical metrics of the patient; determining at least one diagnosis of the patient by applying the CAD module to the most recent clinical metrics of the patient; deriving at least one acuity score for the patient based on the set of diagnosis-specific acuity scores and the determined at least one diagnosis; and displaying, on the display device, the derived at least one acuity score.

One advantage resides in providing a patient status (i.e. patient acuity) score or metric that dynamically detects and aligns with the primary diagnosis of the patient.

Another advantage resides in providing changes to a patient acuity score as more clinical information becomes available, resulting in potential diagnosis changes.

Another advantage resides in associating patient acuity scores having mortality or readmission as an outcome with measurements or clinical actions to be performed in the interim.

Another advantage resides in incorporating physiological trends in determining a patient acuity score.

Another advantage resides in providing a diagnosis-specific acuity score on a common scale independent of the primary diagnosis.

Another advantage resides in providing a patient acuity score that is a blend of diagnosis-specific acuity scores for two (or more) different diagnoses in cases in which the patient has more than one diagnosis (e.g. a diagnosis of AHF and also a diagnosis of AKI).

Another advantage resides in providing a patient acuity score that is a blend of disease-specific acuity scores with the contributions of the constituent diagnosis-specific acuity scores dynamically weighted based on which diagnosis is dominant.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative apparatus for patient acuity monitoring in accordance with the present disclosure.

FIG. 2 diagrammatically illustrates computation of an acuity score using the apparatus of FIG. 1.

FIG. 3 diagrammatically illustrates an example of computation of a blended acuity score using the apparatus of FIG. 1 and employing Bayesian Model Averaging.

FIG. 4 shows an example output by the apparatus of FIG. 1.

FIG. 5 shows an example of possible treatments assigned to different levels of outputs by the apparatus of FIG. 1.

### DETAILED DESCRIPTION

Acuity scores provide a rapid assessment of overall patient health. General-purpose acuity scores such as MEWS or EDI exist. One version of MEWS, for example, assigns points in the range [0,3] for the patient’s systolic blood pressure, heart rate, respiratory rate, temperature, and AVPU (“alert, voice, pain, unresponsive), and totals the points for these five vital signs to generate the MEWS score. There are also diagnosis-specific acuity scores, such as for patients diagnosed with acute heart failure (AHF) or acute kidney injury (AKI). As an example, one standard AKI acuity metric employs criteria operating on serum creatinine and urinary output patient metrics. Serum creatinine is removed by the kidneys, so that elevated serum creatinine is particularly probative of AKI progression; and likewise, urinary output is generated by the kidneys and is therefore also highly probative of AKI progression. Diagnosis-specific acuity score metrics are thus advantageously targeted to the primary diagnosis and therefore may be more sensitive to worsening of the primary diagnosis. However, the specificity of a diagnosis-specific acuity score can make it less effective, or even ineffective, if the patient’s primary diagnosis is different from that for which the diagnosis-specific acuity score is designed. For example, using the just-described AKI acuity score for a patient whose primary diagnosis is AHF may delay detection of a worsening of the AHF, or may even fail to detect worsening of the AHF at all. Selecting the most appropriate diagnosis-specific acuity score is especially challenging in the case of a patient who has two (or more) diseases, such as AHF and AKI. If the patient’s doctor initially identifies AHF as the most critical condition, then an AHF-specific acuity score may be used. But thereafter the patient’s AKI may progressively worsen while the patient’s AHF may be stabilized or even improve. Such a scenario is realistic since the doctor has identified AHF as the critical condition and consequently likely prescribed aggressive AHF treatment. In this case, continued use of the AHF-specific acuity scoring may delay detection of the worsening AKI, or may even miss the worsening AKI completely.

Hence, a problem can arise in that there may be confusion as to which acuity score to employ.

To address this, the following discloses combining a suite of acuity score modules for different diagnoses with a computer aided diagnosis (CAD) module that determines a primary diagnosis of the patient. The CAD module is, in one suitable approach, trained on a set of retrospective patients with clinician-labeled diagnoses. In one embodiment, a single acuity score module is selected from the suite of acuity score modules based on the primary diagnosis output by the diagnosis module, and the acuity score generated by that module is output along with an indication of the primary diagnosis. The suite of acuity score modules comprise a plurality of different diagnosis-specific acuity score modules, e.g. an AHF acuity score module, an AKI acuity score module, and/or so forth. Optionally, the suite of acuity score modules may also include a general-purpose acuity score module, for example implementing MEWS, and the general-purpose acuity score module is selected if no primary diagnosis is determined by the CAD module. As used herein, a general-purpose acuity score module of the suite is considered a diagnosis-specific acuity score module for the diagnosis of “no diagnosis identified” or unsuccessful diagnosis.

In some example embodiments, a blended acuity score is generated. Here the CAD module determines at least one primary diagnosis and may also determine one or more secondary diagnoses, all of which are ranked by probability or likelihood. The acuity scores generated by the acuity score modules corresponding to the primary and secondary diagnoses are then combined as a weighted combination with, for example, the diagnosis probabilities serving as the weights. Here the output is the blended acuity score along with an indication of the primary and secondary diagnoses. This approach may be particularly useful in cases in which the patient may be suffering from two or more different acute conditions (e.g. heart failure and acute kidney injury).

For the blended acuity score approach, the modules of the suite of acuity score modules for the different diagnoses preferably output values using a common scale, e.g. acuity score in the range 0-1 or 0-100% or so forth. In one approach, the acuity score modules of the suite are specially trained for use in the disclosed system, using outcomes of the retrospective patients used in the training.

For the approach in which a single acuity score module for the primary diagnosis is applied, the acuity score modules may be specially trained as just described, or may employ existing (e.g. guideline) acuity scoring criteria. In either case, optionally a converter may be provided with each acuity score module, to convert between the “common units” (e.g. score between 0 and 1) and the guideline-specific score.

In other example embodiments, the features input to the acuity scoring and diagnosis modules may be defined on different time scales. For example, given a heart rate vital sign data stream, one feature might be the current heart rate, while a second feature might be the average heart rate over the last 5 min, a third feature may be the average heart rate over the last 15 minutes, or so forth. This approach of constructing features on different time scales may provide more accurate acuity scoring.

In some examples, if the (near) real-time acuity scoring detects that the primary diagnosis has changed, then an alert may be provided. In other examples, the acuity score may be shown via a trendline.

The disclosed diagnosis-selective acuity scoring is contemplated to be implemented in a patient monitor, and/or at a central (e.g. nurses’) station, and/or at a higher level such as an operational command center. The scoring will use clinical metrics such as the patient’s vital signs (suitably obtained via connection to the patient monitor), and/or information such as bloodwork that may be supplied manually via user input or mined from an Electronic Medical Record (EMR) database.

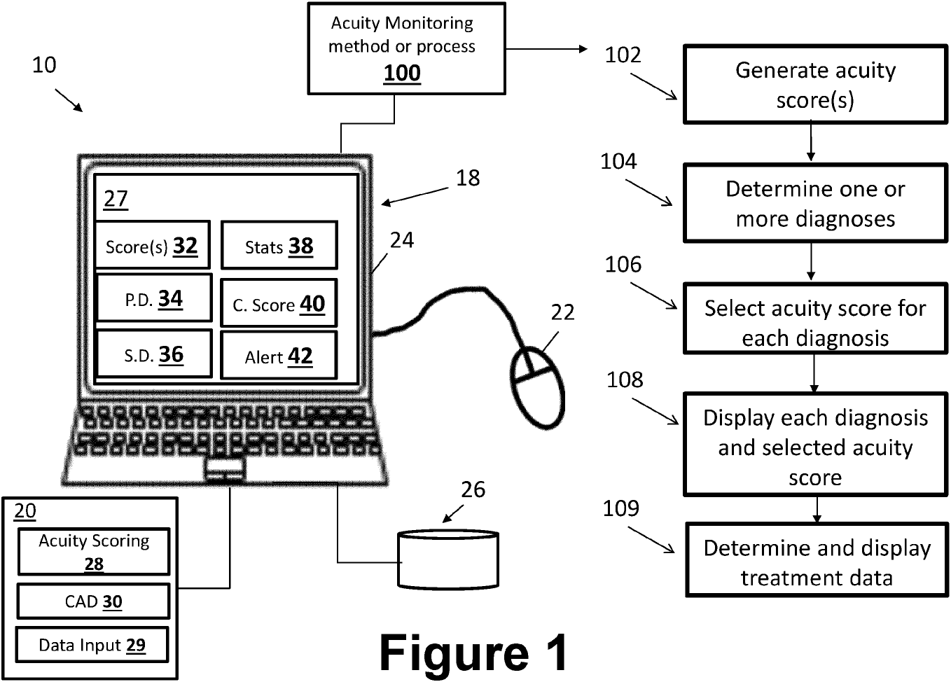

With reference to FIG. 1, an illustrative apparatus 10 is shown for acuity monitoring of a patient. As shown in FIG. 1, the apparatus 10 is depicted as an electronic processing device 18, such as a workstation computer, or more generally a computer, which can be implemented in, for example, a central nurses’ station. However, the apparatus 10 can also be implemented in a patient monitor (e.g., a bedside patient monitor), an operational command center (e.g., a server computer or a plurality of server computers, e.g. interconnected to form a server cluster, cloud computing resource, or so forth, to perform more complex computational tasks). These are merely examples, and should not be construed as limiting. In addition, the apparatus 10 can be operatively connected to one or more databases (not shown), such as an EMR database, an Electronic Health Record (EHR) database, a Radiology Information System (RIS) database, a Picture Archiving and Communication System (PACS) database, and so forth.

As shown in FIG. 1, the illustrative computer 18 includes typical components, such as an electronic processor 20 (e.g., a microprocessor), at least one user input device (e.g., a mouse, a keyboard, a trackball, and/or the like) 22, and a display device 24 (e.g. an LCD display, plasma display, cathode ray tube display, and/or so forth). In some embodiments, the display device 24 can be a separate component from the workstation 18, or may include two or more display devices (e.g., a first display for inputting patient parameters or clinical metrics, and a second display for showing a disease acuity score).

The electronic processor 20 is operatively connected with one or more non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the workstation 18, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a visualization of a graphical user interface (GUI) 27 for display on the display device 24.

As shown in FIG. 1, the electronic processor 20 is programmed to implement multiple processing modules, including one or more diagnosis-specific acuity scoring modules 28, and a computer-aided diagnosis (CAD) module 30. The diagnosis-specific acuity scoring modules 28 are configured to output a corresponding number of patient acuity scores 32 for a corresponding number of potential patient diagnoses.

A given acuity score module 28 may implement an established, e.g. rules-based, acuity scoring algorithm promulgated by a medical association or other entity with domain-specific expertise in that type of diagnosis (for example, the ADHERE model for an AHF acuity score module, a KDIGO model for an AKI acuity score module, or so forth). In another approach, a given acuity score module 28 may implement a diagnosis-specific acuity score algorithm developed using machine learning. The suite of diagnosis-specific acuity scoring modules 28 may optionally include a diagnosis-specific acuity scoring module (for example, implementing MEWS) for the diagnosis of “unsuccessful diagnosis”, that is, for the case in which the CAD module 30 is unable to determine a primary diagnosis for which a diagnosis-specific acuity scoring module 28 is available.

The CAD module 30 is configured to output an indication of at least one primary diagnosis 34 (e.g., each primary diagnosis equally affecting the state of the patient) of the patient from clinical metrics of the patient. Advantageously, the patient acuity scores 32 and the primary diagnosis 34 output by these modules can be combined to provide acuity monitoring of the patient that is diagnosis-specific, but adaptive as the patient’s diagnosis (or diagnoses) change. This monitoring using both of these outputs can be continuous over time, providing diagnosis-adaptive patient acuity monitoring. In addition, the clinical metrics of the patient can be input directly to the acuity scoring modules 28 and the CAD module 30, or can be acquired or received by a data input module 29, e.g. from a patient monitor and/or from an EMR, EHR, or other database containing recorded patient clinical metrics.

The CAD module 30 can be trained with a set of respective patients with clinician-labeled diagnoses. For example, measurements of historical patients, histories of diseases or comorbidities, and admission diagnoses (if available) are used as input features. Each historical patient is labeled according to their primary diagnosis 34. A machine learning model implemented in the CAD module 30 is optimized in order to estimate the primary diagnosis of patients. The output of this process is a model for diagnosis estimation, which can be used to guide selection or combination of relevant acuity scores 32 to display to the care team during hospital stay.

To generate the patient acuity scores 32, the acuity scoring modules 28 can be trained with a set of trained diagnosis focused models. A large set of retrospective patient data is filtered to select for patients with a particular diagnosis. Within this subgroup of patients with the same primary diagnosis 34, time windowed statistics are computed from the features (e.g., clinical metrics for the patients, or features derived from clinical metrics by Principal Component Analysis or other feature extraction techniques). These statistics, as well as the raw feature values, are used to optimize the model for prediction of labeled patient outcomes (such as transition or intervention). The output of the process is a trained acuity scoring model for the particular disease. This process can be repeated for a plurality of diagnoses to generate diagnosis-specific acuity scoring algorithms for the respective acuity scoring modules of the suite of diagnosis-specific acuity scoring modules 28.

In some embodiments, the CAD module 30 is configured to output at least one secondary diagnosis 36 for the patient from clinical metrics of the patient. Advantageously, the apparatus 10 can match corresponding acuity scores 32 to each of the primary diagnosis 34 and the at least one secondary diagnosis 36. The scores 32 can be output individually, or blended to provide an overall acuity score.

Based on the primary diagnosis 34, a corresponding acuity score 32 is selected from a set S of acuity scores. If there are multiple primary diagnoses 34, then a corresponding acuity score 32 is selected for each primary diagnosis. To do so, a patient’s clinical metrics are input to the primary diagnosis model of the CAD module 30. The features are also run through trained models for each potential diagnosis represented by the set S of acuity scores. A model combination algorithm determines how to combine or select from the diagnoses models based on the results of the primary diagnosis model. An output of the model combination algorithm is an acuity score between 0 and 1, and is the probability that the patient may deteriorate. As new data becomes available, the process can be repeated to dynamically update the primary diagnosis 34 and the corresponding acuity score 32.

The apparatus 10 is configured as described above to perform a patient acuity monitoring method or process 100. The non-transitory storage medium 26 stores instructions which are readable and executable by the at least one electronic processor 20 (including the acuity scoring modules 28 and the CAD module 30) to perform disclosed operations including performing the patient acuity monitoring method or process 100. In some examples, the method 100 may be performed at least in part by cloud processing.

With continuing reference to FIG. 1, an illustrative embodiment of the patient acuity monitoring method 100 is diagrammatically shown as a flowchart. To begin the method 100, the data input module 29 can acquire or receive the clinical metrics of the patient. At an operation 102, a set S of diagnosis-specific acuity scores 32 for a corresponding number of a plurality of diagnoses is generated using the diagnosis-specific acuity scoring modules 28 for the respective diagnoses of the plurality of diagnoses applied to the clinical metrics of the patient. In some embodiments, the acuity scoring modules 28 can be trained with a set of respective patients with clinician-labeled diagnoses.

At an operation 104, a primary diagnosis 34 of the patient is determined using the CAD module 30 applied to the clinical metrics of the patient. At an operation 106, the generated acuity score(s) 32 is/are selected for the primary diagnosis 34 from the set S of diagnosis-specific acuity scores. At an operation 108, an indication of the primary diagnosis 34 and the selected acuity score 32 are displayed on the display device 24. The operations 104-108 can be performed in a variety of matters.

In one example embodiment, a single primary diagnosis 34 is determined, and a single acuity score 32 is selected for the primary diagnosis.

In another example embodiment, at least one secondary diagnosis 36 is determined using the CAD module 30, in addition to the primary diagnosis 34. A corresponding acuity score 32 is selected for each determined secondary diagnosis 36, in addition to the primary diagnosis 34. Each secondary diagnosis 36, and the corresponding selected acuity scores 32, are displayed on the display device 24 along with the primary diagnosis 34 and the acuity score selected for the primary diagnosis.

In some examples, diagnosis probabilities 38 can be determined for the primary diagnosis 34 and for each of the generated secondary diagnoses 36 using the CAD module 30 applied to the clinical metrics of the patient. These diagnosis probabilities 38 can also be displayed on the display device 24. The diagnostic probabilities 38 can be computed over past time windows with varying lengths, where longer windows have the greatest memory of past patient status whereas shorter windows consider more recent patient measurements.

In another example embodiment, the acuity scores 32 for the primary diagnosis 34 and each generated secondary diagnosis 36 can be combined to generate a combined acuity score 40, which can be displayed on the display device 24 along with the primary diagnosis 34 and each secondary diagnosis 36. In some examples, the combined acuity score 40 can be generated as a weighted combination with the diagnosis probabilities 38 serving as weights for the weighted combination. In other examples, the weights can be defined by the user, with larger weights given to diagnoses of greater concern.

The operation 108 can be implemented in a variety of examples. In one such example, the acuity scoring modules 28 can output the acuity scores 32 on a common scale, such as a scale ranging from 0-1 (or any other limits), or on a percentage scale of 0-100%, and so forth. If the acuity scoring modules 38 output the acuity scores 32 in different scales, the scores can be converted to a common scale (e.g., all scores are on the 0-1 scale, the 0-100% scale, and so forth). The acuity scores 32 can then be displayed on the display device 24 according to the scale which the scores are generated or converted to. In another example, the selected acuity score 32 for the primary diagnosis 34 (and each acuity score for each secondary diagnosis 36) can be displayed as a trendline.

The operations 102-108 can be repeated over time to provide a continuous acuity monitoring of the patient. For example, the clinical metrics of the patient can be updated as new clinical information about the patient is collected (e.g., vital sign monitoring, physical examination results, imaging examination results, and so forth). The updated (i.e., most recent) clinical metrics are input to the acuity scoring modules 28 to update the acuity scores 32, and to the CAD module 30 to update the primary diagnosis 34 (and each secondary diagnosis 36). If the primary diagnosis 34 is changed as a result of the updated clinical metrics, the display of the primary diagnosis 34 and the corresponding acuity score 32 can be updated in real time (and over time) on the display device 24. In addition, an alert 42 can be output indicting the changing of the displayed primary diagnosis 34. The alert 42 can be output in any suitable manner (e.g., a message displayed on the display device 24, a flashing color on the display device, an audible alert output via a loudspeaker (not shown), an indicating light (not shown) operatively connected to the apparatus 10, and so forth). The updating can be performed continuously over time to provide continuous acuity monitoring of the patient.

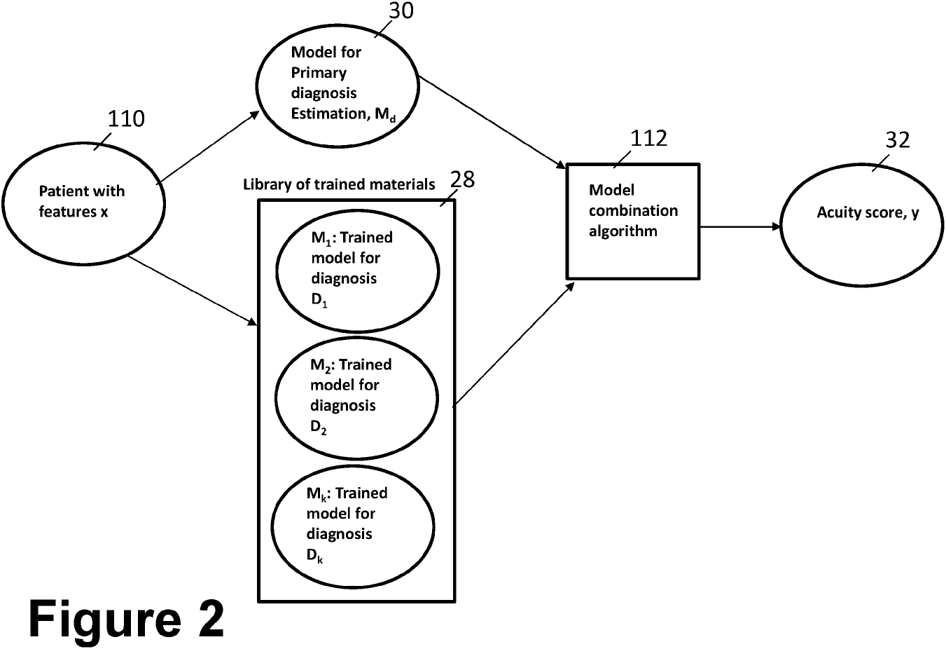

With reference to FIG. 2, a block diagram of the operations 102-108 illustrated as process flow through the modules 28, 30 is shown. Features 110 of a patient to be assessed are input to the suite of diagnosis-specific acuity scoring modules 28 (that is, are input to each of the acuity scoring modules 28 of the suite, corresponding to operation 102) and are input to the CAD module 30 (corresponding to operation 104). A model combination algorithm 112 is applied to the outputs of the acuity scoring modules 28 and the CAD module 30 to output the acuity score (or scores) 32, corresponding to operation 106. In one embodiment, the model combination algorithm 112 selects the acuity score generated by the acuity scoring module 28 corresponding to the primary diagnosis output by the CAD 30. Optionally, this acuity score is labeled by the primary diagnosis when displayed. In another embodiment, the model combination algorithm 112 selects two (or more) scores, e.g. the acuity score generated by the acuity scoring module 28 corresponding to the primary diagnosis output by the CAD 30 and one (or more) acuity score(s) generated by the acuity scoring modules 28 corresponding to the secondary diagnosis (or secondary diagnoses) output by the CAD 30. Preferably, each acuity score is labeled by the primary or secondary diagnosis to which it corresponds when displayed. In yet other embodiments, the model combination algorithm 112 employs a blending algorithm to generate the acuity score 32 as a blended score that combines the acuity scores of the primary diagnosis and at least one secondary diagnosis.

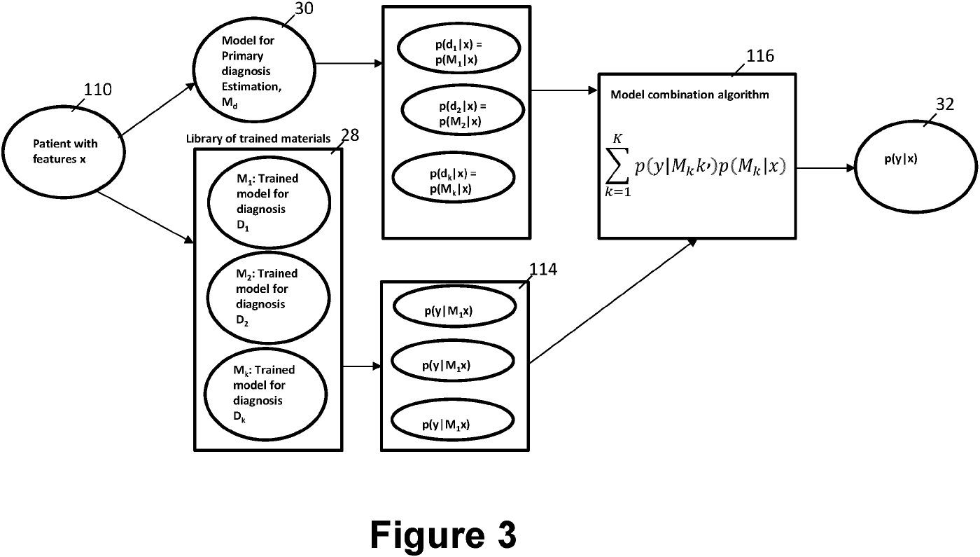

With reference to FIG. 3, an example of a blended scoring embodiment is shown, in which the acuity score is output and updated using an updating process implemented as Bayesian Model Averaging with dynamic updating. Here, the outputs 114 of the respective diagnosis-specific acuity scoring modules 28 are probabilistic outputs, e.g. p(y|M₁, x), p(y|M₂, x), ..., p(y|M_{K}, x), where x denotes the feature vector 110 representing the patient to be assessed, K is the number of acuity scoring modules 28 in the suite of diagnosis-specific acuity modules 28, and M₁ (x), M₂(x), ... , M_{K} (x) denote the acuity models implemented by the respective acuity scoring modules 28. As further shown in FIG. 3, in this embodiment the CAD module 30 outputs diagnosis probabilities for the (candidate) K diagnoses, denoted as p(d₁|x) = p(M₁|x), p(d₂|x) = p(M₂|x), ..., p(d_{K}|x) = p(M_{K}|x). Here dᵢ indicates the diagnosis. The Bayesian Model Averaging 116 then computes the blended acuity score p(y|x) as:

$$p \left(y \vert x\right) = {\sum }_{k = 1}^{K} p \left(y \vert {M}_{k} , k\right) p \left({M}_{k} \vert x\right)$$

With reference back to FIG. 1, when new data for clinical metrics of the patient is available, the model for the primary diagnosis 34 computes the probability 38 p(d₁||x), p(d₂|x), ..., p(d_{K}|x) of each potential diagnosis with the updated clinical metrics. The probability 38 can be considered as the uncertainty for each diagnosis with the highest probability being the most likely primary diagnosis. These probabilities are equivalent to the probability to which acuity scoring modules 28 p(M₁|x), p(M₂|x), ..., p(M_{K}|x) should be implemented. The updated clinical metrics are also run through the trained models to obtain a probability 38 of deterioration (represented in FIG. 3 as the blended acuity score p(y|x)). The probabilities of diagnoses are combined with the conditional model outputs via the model combination algorithm 116, in this case, Bayesian Model Averaging. The output is the probability of deterioration given the updated clinical metrics (again, represented in FIG. 3 as the blended acuity score p(y|x)).

### EXAMPLE

The following describes an application of the method 100. A patient comes to the emergency department with complaints of shortness of breath. A laboratory blood test reveals high levels of NTproBNP, a biomarker indicating that the heart muscle is overworked. The CAD module 30 takes laboratory results and vital signs (e.g., clinical metrics) as input to identify that the patient has acute heart failure (AHF), which is the primary diagnosis 34. The AHF specific model, which had been trained to detect deterioration from a large cohort of AHF patients, is selected to select the corresponding acuity score 32. The AHF acuity score 32 is displayed on the display device 24 (which can be at bedside in the general ward where the patient has been admitted). The acuity scoring modules 28 takes as input quantitative physiologic data, rather than subjective observations, as the clinical metrics in order to reduce variability from observations of different caretakers.

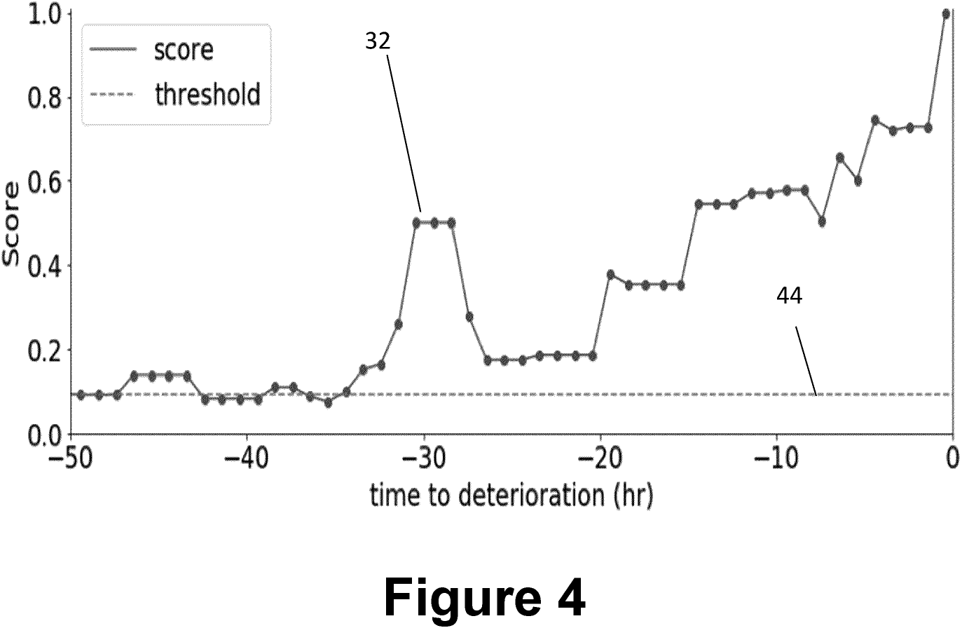

FIG. 4 shows the acuity score 32 of the patient over time. FIG. 4 shows the acuity score 32 as a trendline from time -50 hour to 0 hour (representing the time of transfer to the intensive care unit (ICU)) as compared to a score threshold 44 over time (i.e., the x-axis) over the acuity score (i.e., the y-axis, on an acuity score of 0-1). As shown in FIG. 4, at time -50 hour, the acuity score 32 is close to the threshold 44 (above which the patient would be classified as deteriorating). The acuity score 32 is observed to watch for increases in acuity. At time -35 hour, the acuity score 32 slowly increases. The treating physicians decided to perform a medical intervention, such as increase the dosage of one of the medications in order to stabilize the patient. The care team observes a plateauing of the acuity score 32 followed by a decrease in the acuity score around time -30 hour, indicating that the medication change was effective. At about -20 hour, the patient’s acuity score 32 again begins to slowly increase. Despite additional medication changes and close observation, the acuity does not go down. The care team uses this information to call ahead to the ICU and make sure a bed is available for the patient. The patient is successfully transferred to the ICU at time 0 hour. A cardiology consultant is also scheduled to visit the patient given the patient’s primary diagnosis of acute heart failure.

In some embodiments, the at least one stage value 32 is used to determine treatment data 109 for the patient 12. The treatment data 109 may comprise recommendations for performing varying types of intervention options to treat the patient. For example, the treatment data 109 may be a displayed recommendation to perform a specific pharmaceutical intervention or treatment (e.g., a medication or an intravenous (IV) drip), an imaging session (e.g., ultrasound (US), X-ray, magnetic resonance imaging (MRI), computed tomography (CT), and so forth), a non-imaging diagnostic (e.g., a blood test, a urine test, a pathology test such as a biopsy, etc.), a surgical intervention, or other form of intervention or treatment (e.g., invasive ventilation, cardiac assistive devices, organ transplant, etc.). In some examples, the medical professional may be required to provide an input to the computer 18 to control or otherwise order an intervention option. In another example, the computer 18 may be in communication with an associated drug intervention device to automatically commence a corresponding drug intervention session. In another example, the computer 18 may generate a medical imaging examination order, optionally including information such as imaging parameters for use in the imaging examination. The generated order may be automatically sent to a hospital radiology laboratory to schedule the imaging session, or may be sent to the patient’s physician to review and issue the order. In another example, the computer 18 may generate a non-imaging diagnostic order, optionally including information such as diagnostic parameters.

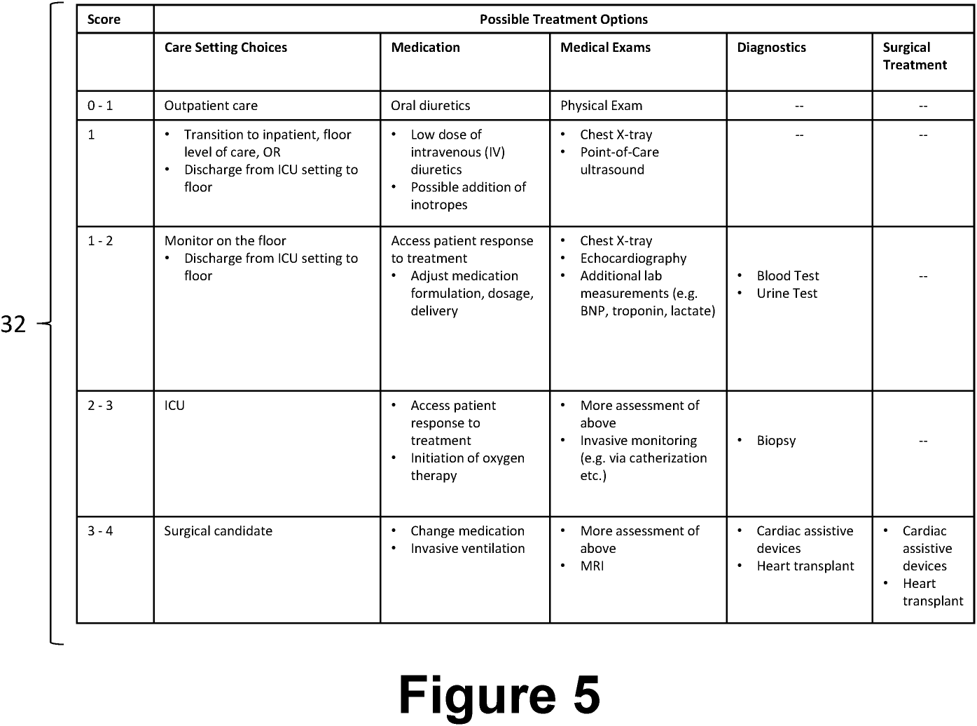

FIG. 5 shows an example of possible treatments or interventions assigned to different levels of acuity scores 32. As shown in FIG. 5, a range of acuity scores 32 can be separated into, for example, 5 levels of possible treatment levels. The 5 levels can include: acuity scores 32 ranging from 0-1; 1; 1-2; 2-3; and 3-4. FIG. 5 shows different medication, medical examination, diagnostics, or surgical treatments associated with each level. A higher level of acuity score is indicative of a higher level of intervention or treatment. For example, a score in the level of 0-1 can include oral diuretics or a physical examination, as opposed to a score in the level of 3-4, in which the interventions or treatments include changing medication, invasive ventilation, cardiac assistive devices, or a heart transplant.

While the apparatus 10 and the method 100 are described primarily in terms of monitoring the acuity score 32 of the patient, the disclosed apparatus and method can be used for other scenarios, such as patient prioritization (e.g., a patient with a higher score is prioritized); earlier planning of transfer to higher acuity settings, notification of discharge readiness from ICU or general ward, evaluation of treatment effectiveness, a metric for quality of life of patient while in-hospital, indicating which patients are candidates for higher acuity interventions (e.g. respiratory ventilation, heart pump, etc.) or for more costly laboratory tests, triggering an alert or alarm that a consultant or specialist needs to be called in for assistance with a particular organ, system, or disease, notifying a regular care team to alter or reconsider course of action because the current approach is putting the patient at risk due to their particular disease or co-morbidity, and comparing acuity of illness from the perspective of different organs or diseases, among others. In some examples, the apparatus 10 and the method 100 enable prioritization of treatments for specific organ systems as the specific risk scores are prioritized over another, especially for patient with comorbidities. In other examples, the apparatus 10 and the method 100 serve as a harmonization mechanism for the various existing risk scores. In other examples, an organ-specific or disease-specific notion of deterioration of a patient can be detected, as opposed to general deterioration, thus providing a more targeted treatment option. In further examples, the acuity scores 32 can be assigned the same as a chosen existing widely-accepted risk score, so that clinicians can more readily associate the scales of the score to established medical knowledge.

Some examples of treatments or interventions have been provided herein solely for the purpose of illustration. One having ordinary skill in the medical arts would understand how the apparatus 10 and method 100 can, in some limiting embodiments, be implemented to provide appropriate or different treatment or intervention recommendations for different types of illnesses which are not described herein.

The apparatus 10 and the method 100, 101 can, in some non-limiting embodiments, be implemented as an improvement to existing commercial products that incorporate disease staging and/or early warning scoring, such as an Intellivue Guardian bedside monitor, or a Central Station (both available from Koninklijke Philips NV, the Netherlands), or any suitable electronic health record system.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing instructions executable by at least one electronic processor to perform a method for staging a disease having a predefined ordered set of S discrete stages where S is an integer having a value greater than or equal to two, the method comprising:
for each discrete stage of the S discrete stages, defining a representative vector for the discrete stage in a vector space defined by a set of clinical metrics based on a set of training patients labeled with the discrete stage and with values for the set of clinical metrics;
for a patient to be staged, receiving patient values for the set of clinical metrics;
generating at least one stage value for the patient to be staged based on distances in the vector space between a patient vector defined in the vector space by the patient values for the set of clinical metrics and the representative vectors for the S discrete stages in the vector space; and
displaying the at least one stage value for the patient to be staged on a display device operatively connected with the electronic processor.

2. The non-transitory computer readable medium of claim 1, wherein, for each discrete stage of the S discrete stages, the defining of the representative vector for the discrete stage includes:
defining training patient vectors in the vector space corresponding to the respective training patients labeled with the discrete stage by the values for the set of clinical metrics labeling the respective training patients; and
defining the representative vector for the discrete stage in the vector space as a centroid of the training patient vectors in the vector space.

3. The non-transitory computer readable medium of claim 1, wherein the generating of the at least one stage value includes:
identifying a closest representative vector of the representative vectors for the S discrete stages for which the distance in the vector space between the patient vector and the representative vector is shortest; and
generating a coarse stage value for the patient to be staged as the discrete stage represented by the closest representative vector, wherein the displaying includes displaying the coarse stage value.

4. The non-transitory computer readable medium of claim 1, wherein the generating of the at least one stage value includes:
identifying the two closest representative vectors of the representative vectors for the S discrete stages that are closest to the patient vector, the two closest representative vectors including a current stage representative vector corresponding to a current stage and a next stage representative vector corresponding to a next stage wherein the current stage is ordered lower than the next stage in the ordered set of S discrete stages; and
generating a fine stage value for the patient to be staged based on the current stage, the next stage, the distance in the vector space between the patient vector and the current stage representative vector, and the distance in the vector space between the current stage representative vector and the next-stage representative vector.

5. The non-transitory computer readable medium of claim 4, wherein the generating of the fine stage value is based on the current stage, the next stage, and a ratio of:
(i) a length of a projection of a current stage-to-patient vector defined as the vector starting at the current stage representative vector and ending at the patient vector onto a current stage-to-next stage vector defined as the vector starting at the current stage representative vector and ending at the next-stage representative vector, and
(ii) a length of the current stage-to-next stage vector.

6. The non-transitory computer readable medium of claim 4, wherein the generating of the fine stage value is based on the current stage, the next stage, and a ratio:
$$\frac{\left‖{D}_{N \to P a}\right‖}{\left‖{D}_{N \to N + 1}\right‖}$$
where ||D_{N→N+1}|| is the length of a vector D_{N→N+1} from the current stage representative vector to the next stage representative vector and D_{N→Pa} is a vector given by the dot product:
$${D}_{N \to P} ⋅ \frac{{D}_{N \to N + 1}}{\left‖{D}_{N \to N + 1}\right‖}$$
where D_{N→P} is a vector from the current stage representative vector to the patient vector.

7. The non-transitory computer readable medium of claim 4, wherein the current stage is assigned a first integer value, the next stage is assigned a second integer value different from the first integer value, and the fine stage value is a real number lying between the first integer value and the second integer value.

8. The non-transitory computer readable medium of claim 1, wherein the distances in the vector space are computed using a distance function selected from a group including a Euclidean distance function, a Hamming distance function, a Geometric distance function, and a cosine distance function.

9. The non-transitory computer readable medium of claim 1, wherein the method further includes:
automatically assigning the discrete stage labels to the training patients using a deterministic staging algorithm based on values of a subset of the set of clinical metrics wherein the deterministic staging algorithm assigns a discrete stage selected from the predefined ordered set of S stages.

10. The non-transitory computer readable medium of claim 1, wherein the method further includes:
selecting the set of clinical metrics from a superset of clinical metrics using an automated feature selection algorithm.

11. The non-transitory computer readable medium of claim 1, wherein the method further includes associating the at least one stage value to treatment data comprising at least one intervention option to treat the patient, and the method further includes at least one of:
displaying the treatment data; and
commencing at least one treatment option to treat the patient based on the treatment data.

12. An apparatus for staging a disease having a predefined ordered set of S discrete stage where S is an integer having a value greater than or equal to two, the apparatus comprising:
at least one electronic processor; and
a non-transitory computer readable medium storing instructions readable and executable by at least one electronic processor to perform a method including:
for a patient to be staged, receiving patient values for a set of clinical metrics;
using the received patient values, defining a patient vector in a vector space defined by the set of clinical metrics;
generating at least one stage value for the patient to be staged based on distances in the vector space between the patient vector and representative vectors in the vector space that represent respective discrete stages of the predefined ordered set of S discrete stages; and
controlling a display device operatively connected with the electronic processor to display the at least one stage value for the patient to be staged.

13. The apparatus of claim 12, wherein the method further includes:
for each discrete stage of the S discrete stages, defining a representative vector for the discrete stage in a vector space defined by a set of clinical metrics based on a set of training patients labeled with the discrete stage and with values for the set of clinical metrics.

14. The apparatus of claim 13, wherein, for each discrete stage of the S discrete stages, the defining of the representative vector for the discrete stage includes:
defining training patient vectors in the vector space corresponding to the respective training patients labeled with the discrete stage by the values for the set of clinical metrics labeling the respective training patients; and
defining the representative vector for the discrete stage in the vector space as a centroid of the constructed training patient vectors in the vector space.

15. The apparatus of claim 13, wherein the generating of the at least one stage value includes:
identifying a closest representative vector of the representative vectors for the S discrete stages for which the distance in the vector space between the patient vector and the representative vector is shortest; and
generating a coarse stage value for the patient to be staged as the discrete stage represented by the closest representative vector, wherein the displaying includes displaying the coarse stage value.

16. The apparatus of claim 13, wherein the generating of the at least one stage value includes:
identifying two closest representative vectors of the representative vectors for the S discrete stages which are closest to the patient vector, the two closest representative vectors including a current stage representative vector corresponding to a current stage and a next stage representative vector corresponding to a next stage wherein the current stage is ordered lower than the next stage in the ordered set of S discrete stages; and
generating a fine stage value for the patient to be staged based on the current stage, the next stage, the distance in the vector space between the patient vector and the current stage representative vector, and the distance in the vector space between the current stage representative vector and the next-stage representative vector.

17. The apparatus of claim 16, wherein the generating of the fine stage value is based on the current stage, the next stage, and a ratio of:
(i) the length of a projection of a current stage-to-patient vector defined as the vector starting at the current stage representative vector and ending at the patient vector onto a stage-to-next stage vector defined as the vector starting at the current stage representative vector and ending at the next stage representative vector, and
(ii) the length of the stage-to-next stage vector.

18. The apparatus of claim 12, wherein the method further includes:
automatically assigning the discrete stage labels to the training patients using a deterministic staging algorithm based on values of a subset of the set of clinical metrics wherein the deterministic staging algorithm assigns a discrete stage selected from the predefined ordered set of S stages.

19. The apparatus of claim 12, wherein the method further includes:
selecting the set of clinical metrics from a superset of clinical metrics using an automated feature selection algorithm.

20. A method for staging a disease having a predefined ordered set of S discrete stages where S is an integer having a value greater than or equal to two, the method comprising:
for each discrete stage of the S discrete stages, defining a representative vector for the discrete stage in a vector space defined by a set of clinical metrics based on a set of training patients labeled with the discrete stage and with values for the set of clinical metrics by operations including:
defining training patient vectors in the vector space corresponding to the respective training patients labeled with the discrete stage by the values for the set of clinical metrics labeling the respective training patients; and
defining the representative vector for the discrete stage in the vector space as a centroid of the constructed training patient vectors in the vector space;
for a patient to be staged, receiving patient values for the set of clinical metrics;
generating at least one stage value for the patient to be staged based on distances in the vector space between a patient vector defined in the vector space by the patient values for the set of clinical metrics and the representative vectors for the S discrete stages in the vector space; and
displaying the at least one stage value for the patient to be staged on a display device.
